(19) United States
(12) Patent Application Publication
(10) Pub. No.: US 2017/0281509 A1
(43) Pub. Date: Oct. 5, 2017

(54) COMPOSITIONS AND METHODS FOR MITIGATING SKIN IRRITATION

(71) Applicant: GOJO INDUSTRIES, INC., Akron, OH (US)

(72) Inventors: Christopher A. Wolf, Ashland, OH (US); Jessica R. Tittl, Akron, OH (US)

(73) Assignee: GOJO INDUSTRIES, INC., Akron, OH (US)

(21) Appl. No.: 15/509,670

(22) PCT Filed: Sep. 14, 2015
(86) PCT No.: PCT/US2015/049890

§ 371 Date: Mar. 8, 2017

Related U.S. Application Data
(60) Provisional application No. 62/049,649, filed on Sep. 12, 2014.

(51) Int. Cl. A61K 8/64; A61K 8/06; A61K 8/37; A61Q 19/10; A61K 8/92; A61K 8/97

(52) U.S. Cl. CPC A61K 8/64; A61K 8/922; A61K 8/97; A61K 8/375; A61Q 19/10; A61K 8/06; A61K 2800/75

(57) ABSTRACT

Methods and compositions for decreased irritation of mammalian skin cells are provided. Compositions contain at least one amide-functionalized oligopeptide. The compositions may advantageously be applied to mammalian skin to effect a reduction in inflammation and redness experienced by the skin, and/or to reduce skin irritation.

### RELATED CASES

This application claims the benefit of U.S. Provisional Ser. No. 62/049,649, filed on Sep. 12, 2014, which is incorporated herein by reference.

### TECHNICAL FIELD

One or more embodiments of the present invention provide methods and compositions for mitigating the irritation of mammalian skin cells. More particularly, it relates to compositions containing at least one amide-functionalized oligopeptide and an acceptable carrier. The compositions can be applied to mammalian skin to reduce the inflammation and redness that can result from the use of skin irritants.

### BACKGROUND OF THE INVENTION

Many ingredients in skin care and cosmetic products can cause skin irritation. Surfactants such as sodium lauryl sulfate (SLS) are known to be skin irritants. Retinoid and its derivatives, can cause severe local irritation manifested as mild erythema and stratum corneum peeling of the skin. Topical or systemic use of some skin cleansers and disinfectants is linked to skin irritation.

Ingredients such as benzyol peroxide, alpha-hydroxyl acids and derivatives thereof, salicylic acid, natural plant extracts, sunscreen actives, urea, and preservatives are also known to cause external skin irritations. Furthermore, skin irritations may be caused by inherent disease conditions such as acne, rosacea, atopic dermatitis, and other disease states. Typical approaches to reduce irritation include reducing the concentration of the inflammatory ingredient, use of alternatives or formulation/delivery approaches, such as encapsulation, controlled release, compartmentalization, inclusion of non-irritating excipients. None of the above has successfully reduced irritation while retaining efficacy. As a result, there is a need for anti-irritant substances to mitigate external skin irritations, or irritations caused by inherent skin conditions.

### SUMMARY OF THE INVENTION

One or more embodiments of the present invention provide a composition comprising at least one amide-functionalized oligopeptide.

One or more embodiments of the present invention further provide a method for the treatment of the skin to condition the skin and/or to reduce irritation, the method comprising the step of contacting the skin with a composition comprising at least one amide-functionalized oligopeptide and a cosmetically or pharmaceutically acceptable carrier.

One or more embodiments of the present invention further provide a method for the cleansing and/or sanitizing skin, the method comprising the steps of combining a skin cleansing or skin sanitizing composition with one or more amide-functionalized oligopeptides, wherein the amide-functionalized oligopeptide reduces the irritancy potential of the composition; and contacting the skin with the reduced-irritancy composition in a manner sufficient to cleanse and/or sanitize the skin.

One or more embodiments of the present invention further provide a method for reducing the irritancy potential of a skin cleanser or skin sanitizer composition, the method comprising the steps of combining a skin cleans or skin sanitizer composition with one or more amide-functionalized oligopeptides, wherein the amide-functionalized oligopeptide reduces the irritancy potential of the composition.

### BRIEF DESCRIPTION OF THE DRAWINGS

FIG. 1 is a graphical representation of the effect of compositions containing amide-functionalized oligopeptides on the irritation response of cells treated with known irritants, as quantified by measuring IL-8 secretion.

FIG. 2 is a graphical representation of the reduction of IL-8 secretion for test samples, compared to Control B, which contained no amide-functionalized oligopeptide.

### DETAILED DESCRIPTION OF ILLUSTRATIVE EMBODIMENTS

In one or more embodiments, compositions of the present invention include at least one amide-functionalized oligopeptide, and a cosmetically or pharmaceutically acceptable carrier. In one or more embodiments, the carrier is a lotion formulation that includes a vegetal wax, and olive derivative or extract, and optionally, an emulsifier.

Generally, oligopeptides are short chains of amino acid moieties linked by amide bonds. Any combination of amino acids may be included. Amide-functionalized oligopeptides are oligopeptides that contain at least one amide functional group.

In one or more embodiments, the amide-functionalized oligopeptide includes 12 amino acid moieties or less, in other embodiments, 10 amino acid moieties or less, and in other embodiments, 8 amino acid moieties or less. In one or more embodiments, the peptide includes at least 6 amino acid moieties. In one or more embodiments, the peptide includes 6 amino acid moieties.

In one or more embodiments, compositions of the present invention include at least one amide-functionalized acetyl hexapeptide. In one or more embodiments, the amide-functionalized oligopeptide may be identified by an INCI (International Nomenclature of Cosmetic Ingredients) name, such as acetyl hexapeptide-X amide, where X is a numerical designation such as 51.

Some non-limiting examples of acetyl hexapeptides that contain at least one functional group include acetyl hexpapetide-51 amide.

Acetyl hexapeptide-51 amide is commercially available, for example from Lipotec LLC as a blend with butylene glycol and water, under the tradename Juveleven™.

In one or more embodiments, compositions of the present invention further comprise a cosmetically or pharmaceutically acceptable carrier. In one or more embodiments, the term carrier relates to a diluent, adjuvant, or excipient. In one or more embodiments, suitable carriers include liquids, such as water, oils or surfactants.

Suitable carriers also include solids, liquids, or semisolid formulations, such as creams, multiple emulsions, such as oil and/or silicone in water emulsions, water-in-oil and/or silicone emulsions, water/oil/water or water/silicone/water type emulsions, and oil/water/oil or silicone/water/silicone type emulsions, anhydrous compositions, aqueous dispersions, oils, milks, balsams, foams, lotions, gels, cream gels, hydroalcoholic solutions, hydroglycolic solutions, hydrogels, liniments, sera, soaps, shampoos, conditioners, serums, polysaccharide films, ointments, mousses, pomades, powders, bars, pencils and sprays or aerosols, including leave-on and rinse-off formulations.

Advantageously, the amide-functionalized oligopeptide can be employed in a wide variety of topical products in order to mitigate the irritancy of the product, or to enhance the skin-conditioning benefits of the product. Topical products in which the amide-functionalized oligopeptide may be employed include aqueous or alcohol-based sanitizers, surfactant-based washes, lotions, serums, shampoos and conditioners, creams, aqueous based leave on gels, sprayable sanitizers, sprayable lotions, sunscreens, after sun care, eye creams, lip products.

In one or more embodiments, compositions of this invention are in the form of topical products that can be applied externally to the skin.

Suitable topical products include aqueous or alcohol-based sanitizer compositions, such as those described in U.S. Pat. Nos. 4,956,170, 6,143,710, 6,183,766, 6,228,385, 6,248,343, 7,803,390, 8,293,802, 8,323,633, 8,329,758, 8,338,491, 8,697,103, all of which are hereby incorporated by reference.

Suitable topical products also include surfactant-based sanitizer compositions, such as those described in U.S. Pat. Nos. 5,712,232, 5,972,860, 6,479,442, 6,413,921, 7,517,842, and 8,372,790, all of which are hereby incorporated by reference.

Suitable topical products also include lotions. Examples of lotion formulations include those containing water and/or alcohols and emollients such as hydrocarbon oils and waxes, silicone oils, hyaluronic acid, vegetable, animal or marine fats or oils, glyceride derivatives, fatty acids or fatty acid esters or alcohols or alcohol ethers, lanolin and derivatives, polyhydric alcohols or esters, wax esters, sterols, phospholipids and the like, and generally also emulsifiers (nonionic, cationic or anionic), although some of the emollients inherently possess emulsifying properties.

In one or more embodiments, the amide-functionalized oligopeptide may be incorporated into a sustained release system that provides the gradual release of this compound during a period of time.

In one or more embodiments, the carrier comprises a lotion having a liquid crystalline network structure. Suitable lotion carriers are described in co-pending U.S. Provisional Patent Application No. 62/140,542, filed on Mar. 31, 2015, which is hereby incorporated by reference.

In one or more embodiments, the compositions of the present invention may be formulated as lotions. As is known in the art, lotions include oil-in-water emulsions as well as water-in-oil emulsions, oil-water-oil, and water-oil-water. A wide variety of ingredients may be present in either the oil or water phase of the emulsion. That is, the lotion formulation is not particularly limited.

Examples of lotion formulations include those containing water and/or alcohols and emollients such as hydrocarbon oils and waxes, silicone oils, hyaluronic acid, vegetable, animal or marine fats or oils, glyceride derivatives, fatty acids or fatty acid esters or alcohols or alcohol ethers, lanolin and derivatives, polyhydric alcohols or esters, wax esters, sterols, phospholipids and the like, and generally also emulsifiers (nonionic, cationic or anionic), although some of the emollients inherently possess emulsifying properties.

These same general ingredients may be formulated into a cream rather than a lotion, or into gels, or into solid sticks by utilization of different proportions of the ingredients and/or by inclusion of thickening agents such as gums, carbomers, or other forms of hydrophilic colloids. Generally, creams and ointments may be typically spreadable in the range from room temperature to skin temperature, and lotions and milks may be more flowable within this temperature.

In one or more embodiments, the carrier is water-based. In one or more embodiments, the carrier contains one or more vegetal waxes, one or more olive derivatives or extracts, AND one or more emulsifiers.

In one or more embodiments, the carrier includes a vegetal wax. Advantageously, the vegetal wax promotes a liquid crystal network structure to the composition.

In one or more embodiments, a liquid crystal network structure in the compositions of the present invention improves the physiological penetration of active ingredients, increases the barrier integrity, and aids in functional hydration.

Without wishing to be bound by theory, it is believed that carriers including vegetal wax according to the present invention provide a complex combination of fatty acids that are chemically similar to the skin surface lipid composition, and that have the distinctive property to self-emulsify in hydrophilic or lipophilic millieus. In one or more embodiments, the distinctive complex combination of fatty acids represent a unique biomimetic restructuring agent endowed with the double feature of first restoring and maintaining the integrity of the skin barrier and then providing itself the emulsifying base.

Compositions of the present invention containing a liquid crystalline carrier provide liquid crystals with a skin-like fatty acid composition. The highly stable and dermo-compatible liquid crystals are similar to the lipids of the cutaneous barrier. In one or more embodiments, the liquid crystals act as biomimetic restructuring agents and restore the optimal integrity of the skin barrier function and increase the integrity of the stratum corneum barrier function leading to an increased and sustained skin hydration.

The vegetal wax is a vegetable-based liquid crystal promoter that is believed to stabilize oil-in-water emulsions and to improve the texture of emulsions. As a liquid crystal promoter, the vegetal wax re-organizes the emulsion's microscopic structure, acting as an emulsion stabilizing agent. The lamellar liquid crystals produce several bi-layers that enrobe the oil droplets, producing an energy layer preventing coalescence.

In one or more embodiments, the vegetal wax provides the lotion with sebum-control benefits. In one or more embodiments, it provides skin-hydration and a unique texture due to the high water content of the liquid crystalline structure (water incorporated between several bilayers). In one or more embodiments, the vegetal wax influences positively the delivery of active ingredients to the skin.

Examples of vegetal waxes include a blend of cetyl palmitate, sorbitan palmitate and sorbitan olivate such as the blend that is available from B&T S.r.l. under the tradename Oliwax LC.

In one or more embodiments, the composition further comprises at least one derivative or extract of olives. In one or more embodiments, the olive derivative includes olive oil. In one or more embodiments, the olive derivative includes a cetearylic ester derivative and/or a sorbitan ester derivative. In one or more embodiments, the olive derivative includes a blend of a cetearylic ester derivative and a sorbitan ester derivative. In one or more embodiments, the olive derivative includes a blend having an INCI designation of Cetearyl Olivate (and) Sorbitan Olivate. Cetearyl olivate (and) sorbitan olivate is available from B&T S.r.l. under the tradename OliveM®1000. OliveM is sometimes described as an O/W emulsifier derived from olive oil. It is substantially free of polyethylene oxides (PEG).

In one or more embodiments, it is believed that the cetearylic ester derivative stabilizes the liquid crystals. In certain embodiments, it is believed that the sorbitan ester derivative enhances the emolliency properties of the composition and/or provides easier dispersion for powders. Advantageously, UV filters and pigments may be easily dispersed at high percentages. It is believed that the blend of a cetearylic ester derivative and a sorbitan ester derivative of olive combines a liquid crystal structure with an oleic component derived from olive oil. In one or more embodiments, skin penetration is enhanced, and a soft, silky smooth after-feel is obtained. The substantivity of its composition, being very similar to the human sebum, provides retention of the skin moisture and increases the active ingredient's resistance to water and/or sweat.

It is believed that the blend of a cetearylic ester derivative and a sorbitan ester derivative of olive operates by forming liquid crystals in emulsions, by placing itself at the interface of a two phase system in a preferential direction, placing the polar head into the aqueous phase and the nonpolar tail into the lipidic phase. In one or more embodiments, the postmicellar organization of the blend of a cetearylic ester derivative and a sorbitan ester derivative of olive in water is the typical structure of a liquid crystal reticule, where the bilayer micelles create a multilayer lamellar formation. Below are images of the formation first of lamellar liquid crystals (Picture A), and of the reticular structure (Picture B)

In one or more embodiments, emulsions that are formulated with the blend of a cetearylic ester derivative and a sorbitan ester derivative of olive appear very shiny and light and have an original, fresh and silky touch, even if they contain high percentages of lipids. The blend of a cetearylic ester derivative and a sorbitan ester derivative of olive, promoting the formation of this reticular structure inside the emulsion, allows the formulation to contain quite large amounts of natural and polar lipids without affecting the final stability of the emulsion. In one or more embodiments, up to about 25 wt. % of the emulsion may be natural and/or polar lipids.

In one or more embodiments, the carrier includes one or more emulsifiers. Examples of emulsifiers include glycerol esters, in particular glycerol esters of α-hydroxycarboxylic acids and saturated fatty acids. Specific examples include glyceryl stearate.

Compositions or the present invention may further comprise one or more of a wide range of optional ingredients, with the proviso that they do not deleteriously affect the beneficial properties of the composition. The CTFA International Cosmetic Ingredient Dictionary and Handbook, Twelfth Edition 2008, and the 2007 CTFA International Buyer's Guide, both of which are incorporated by reference herein in their entirety, describe a wide variety of non-limiting cosmetic and pharmaceutical ingredients commonly used in the skin care industry, that are suitable for use in the compositions of the present invention. Examples of optional ingredients, classified by function, include: abrasives, anti-acne agents, anticaking agents, antioxidants, binders, biological additives, bulking agents, chelating agents, chemical additives; colorants, cosmetic astringents, cosmetic biocides, denaturants, drug astringents, emulsifiers, external analgesics, film formers, fragrance components, humectants, opacifying agents, plasticizers, preservatives (sometimes referred to as antimicrobials), propellants, reducing agents, skin bleaching agents, skin-conditioning agents (emollient, miscellaneous, and occlusive), skin protectants, solvents, surfactants, foam boosters, hydrotropes, solubilizing agents, suspending agents (nonsurfactant), sunscreen agents, ultraviolet light absorbers, detackifiers, and viscosity increasing agents (aqueous and nonaqueous). Examples of other functional classes of materials useful herein that are well known to one of ordinary skill in the art include solubilizing agents, sequestrants, and keratolytics, topical active ingredients, and the like. One or more optional benefit agents selected from the group consisting of primary skin-conditioning agents, deposition enhancers, humectants, moisturizing esters, emulsifying agents, silicone glycols, miscellaneous skin conditioners, thickeners, and/or antimicrobial agents.

In one or more embodiments, compositions of the present invention comprise at least an effective amount of the amide-functionalized oligopeptide, wherein an effective amount is the amount that mitigates skin irritation, when compared to the same composition but not containing any amide-functionalized oligopeptide. In one or more embodiments, an effective amount of amide-functionalized oligopeptide is at least about 0.06 parts per million by weight (ppm), based upon the total weight of the composition. In other embodiments, an effective amount is at least about 0.10 ppm, in other embodiments, at least about 0.12 ppm, based upon the total weight of the composition.

In one or more embodiments, the composition comprises from about 0.06 to about 100 ppm of amide-functionalized oligopeptide, based upon the total weight of the composition. In one or more embodiments, the composition comprises from about 0.08 to about 50 ppm of amide-functionalized oligopeptide, based upon the total weight of the composition. In one or more embodiments, the composition comprises from about 0.1 to about 30 ppm of amide-functionalized oligopeptide, based upon the total weight of the composition. In one or more embodiments, the composition comprises from about 0.5 to about 25 ppm of amide-functionalized oligopeptide, based upon the total weight of the composition.

In one or more embodiments, the composition includes at least one vegetal wax in an amount of at least about 0.5 weight percent (wt. %), in other embodiments, at least about 0.75 wt. %, in other embodiments, at least about 1 wt. %, in other embodiments, at least about 1.5 wt. %, in other embodiments, at least about 2 wt. %, in other embodiments, at least about 2.5 wt. %, based upon the total weight of the composition.

In one or more embodiments, the composition includes at least one vegetal wax in an amount of up to about 10 weight percent (wt. %), in other embodiments, up to about 8 wt. %, in other embodiments, up to about 5 wt. %, in other embodiments, up to about 3 wt. %, in other embodiments, up to about 2.5 wt. %, in other embodiments, up to about 2 wt. %, in other embodiments, up to about 1.5 wt. %, in other embodiments, up to about 1 wt. %, based upon the total weight of the composition.

In one or more embodiments, the composition includes at least one vegetal wax in an amount of from about 0.5 to about 10 wt. %, in other embodiments, from about 0.75 to about 8 wt. %, and in other embodiments, from about 1 to about 5 wt. %, based upon the total weight of the composition.

In one or more embodiments, the composition includes at least one olive derivative or extract in an amount of at least about 0.1 wt. %, in other embodiments, at least about 0.25 wt. %, in other embodiments, at least about 0.5 wt. %, in other embodiments, at least about 0.75 wt. %, in other embodiments, at least about 1 wt. %, in other embodiments, at least about 1.5 wt. %, in other embodiments, at least about 2 wt. %, in other embodiments, at least about 2.5 wt. %, based upon the total weight of the composition.

In one or more embodiments, the composition includes at least one olive derivative or extract in an amount of up to about 20 wt. %, in other embodiments, up to about 18 wt. %, in other embodiments, up to about 15 wt. %, in other embodiments, up to about 10 wt. %, in other embodiments, up to about 8 wt. %, in other embodiments, up to about 5 wt. %, in other embodiments, up to about 3 wt. %, in other embodiments, up to about 2 wt. %, in other embodiments, up to about 1 wt. %, in other embodiments, up to about 0.5 wt. %, based upon the total weight of the composition.

In one or more embodiments, the composition includes at least one olive derivative or extract in an amount of from about 0.1 to about 20 wt. %, and in other embodiments, from about 0.5 to about 15 wt. %, based upon the total weight of the composition.

In one or more embodiments, the composition includes at least one olive oil in an amount of at least about 0.1 wt. %, in other embodiments, at least about 0.25 wt. %, in other embodiments, at least about 0.5 wt. %, in other embodiments, at least about 0.75 wt. %, in other embodiments, at least about 1 wt. %, in other embodiments, at least about 1.5 wt. %, in other embodiments, at least about 2 wt. %, in other embodiments, at least about 2.5 wt. %, based upon the total weight of the composition.

In one or more embodiments, the composition includes at least one olive oil in an amount of up to about 20 wt. %, in other embodiments, up to about 18 wt. %, in other embodiments, up to about 15 wt. %, in other embodiments, up to about 10 wt. %, in other embodiments, up to about 8 wt. %, in other embodiments, up to about 5 wt. %, in other embodiments, up to about 3 wt. %, in other embodiments, up to about 2 wt. %, in other embodiments, up to about 1 wt. %, in other embodiments, up to about 0.5 wt. %, based upon the total weight of the composition.

In one or more embodiments, the composition includes at least one olive oil in an amount of from about 0.1 to about 20 wt. %, in other embodiments, from about 0.5 to about 15 wt. %, based upon the total weight of the composition.

In one or more embodiments, the total amount of the emulsifiers in the composition is from about 0.1 to about 10.0 wt. %, in one or more embodiments, from about 0.5 to about 6.0 wt. %, based on the total weight of the composition.

In one or more embodiments, the total amount of the glycerol esters in the composition is from about 0.1 to about 10.0 wt. %, in one or more embodiments, from about 0.5 to about 6.0 wt. %, based on the total weight of the composition.

In one or more embodiments, strong acids and strong bases or other ingredients that may attack the peptide bonds in the amide-functionalized oligopeptide may be limited. More specifically, in one or more embodiments, the amount of strong acids is less than about 0.5 percent by weight, in another embodiment, less than about 0.1 percent by weight, in another embodiment, less than about 0.05 percent by weight, based upon the total weight of the composition. In another embodiment, the composition is devoid of strong acid. In one or more embodiments, the amount of strong bases is less than about 0.5 percent by weight, in another embodiment, less than about 0.1 percent by weight, in another embodiment, less than about 0.05 percent by weight, based upon the total weight of the composition. In another embodiment, the composition is devoid of strong base.

In one or more embodiments, the amount of protein denaturants is limited. In one or more embodiments, the amount of protein denaturants is less than about 5 percent by weight, in another embodiment, less than about 1 percent by weight, in another embodiment, less than about 0.5 percent by weight, based upon the total weight of the composition. In another embodiment, the composition is devoid of protein denaturants.

In one or more embodiments, elevated temperatures are avoided.

The composition may be prepared by simply mixing the components together. The order of addition is not particularly limited, but may advantageously be selected based upon the solubility of the various ingredients.

The amide-functionalized oligopeptides of this invention may have variable solubility in water. Water-soluble amide-functionalized oligopeptides may be incorporated directly into aqueous compositions. Water-insoluble amide-functionalized oligopeptides and those with limited water solubility may be solubilized in cosmetically or pharmaceutically acceptable solvents such as and not restricted to, ethanol, propanol, isopropanol, propylene glycol, glycerin, butylene glycol or polyethylene glycol or any combination thereof.

Thus, in one or more embodiments, the amide-functionalized oligopeptide may be added to the composition as a solution or emulsion. In other words, the amide-functionalized oligopeptide may be premixed with a solvent, and optionally one or more other ingredients, to form an amide-functionalized oligopeptide solution or emulsion, with the proviso that the other ingredients do not deleteriously affect the beneficial properties of the composition.

Examples of carriers include water, alcohol, or blends of water and another carrier such as glycols, ketones, linear and/or cyclic hydrocarbons, triglycerides, carbonates, silicones, alkenes, esters such as acetates, benzoates, fatty esters, glyceryl esters, ethers, amides, polyethylene glycols, PEG/PPG copolymers, inorganic salt solutions such as saline, and mixtures thereof. For example, in one or more embodiments, the amide-functionalized oligopeptide is pre-blended with butylene glycol and water. In one or more embodiments, the amount of amide-functionalized oligopeptide in the blend is about 0.05 wt. %, based upon the total weight of the blend. It will be understood that, when the amide-functionalized oligopeptide is premixed to form an amide-functionalized oligopeptide solution or emulsion, the amount of solution or emulsion that is added to the composition is selected so that the amount of amide-functionalized oligopeptide falls within the ranges set forth hereinabove.

In one or more embodiments, the composition is topically applied to skin. In one or more embodiments, the composition may be topically applied to an affected skin area in a predetermined or as-needed regimen. In one or more embodiments, the composition is included as part of a skin cleansing or sanitizing regimen.

Advantageously, in one or more embodiments, skin cleansers, sanitizers and lotions containing one or more amide-functionalized oligopeptide according to the present invention have a reduced irritancy potential, when compared to the same skin cleanser, sanitizer or lotion but not containing one or more amide-functionalized oligopeptide according to the present invention.

Thus, the present invention further provides a method for reducing the irritancy potential of a skin cleanser or sanitizer. The method includes the step of combining a skin cleanser or sanitizer composition with one or more amide-functionalized oligopeptides to form a less irritating skin cleanser or sanitizer composition. The method includes the further step of contacting the skin with the less irritating composition for a period sufficient to cleanse and/or sanitize the skin. In one or more embodiments, when the amount of skin irritation is measured, as for example by testing the IL-8 secretion, the amount of skin irritation is reduced, compared to when the method is repeated but using the same skin cleanser or sanitizer composition without any amide-functionalized oligopeptide.

Alternatively, a sequential method may be employed. Thus, in one or more embodiments, the present invention further provides a method for cleansing and/or sanitizing skin. In a first step of the method, a skin cleanser or sanitizer composition is provided, and mammalian skin is contacted with the skin cleanser or sanitizer composition in an amount and for a time suitable for cleansing or sanitizing the skin. In a second step, a composition comprising at least one amide-functionalized oligopeptide (referred to as a second composition) is provided, and the cleansed or sanitized skin is contacted with the second composition, wherein the second composition mitigates the skin irritation that might have been caused by the skin cleanser or sanitizer composition. The second composition may be formulated as a leave-on product, or may optionally be rinsed off of the skin.

Advantageously, compositions and methods of the present invention may be useful to treat a variety of skin conditions that result in inflammation or erythema. For example, inflammation or erythema can result from external causes such as sun or wind burn or irritating soaps or cleansers. It is also known that inflammation and erythema can be caused from inherent conditions such as rosacea, atopic dermatitis, or allergic skin reactions.

In order to demonstrate the practice of the present invention, the following examples have been prepared and tested. The examples should not, however, be viewed as limiting the scope of the invention. The claims will serve to define the invention.

### EXAMPLES

Interleukin 8 (IL-8) is a chemokine and proinflammatory cytokine produced by macrophages and other cell types such as epithelial cells. It is secreted from keratinocytes in skin in response to inflammatory stimuli. IL-8 is secreted and is an important mediator of the immune reaction in the innate immune system response. IL-8 overexpressed is a biomarker of skin irritation. Thus, the following method may be employed to measure the irritancy potential of an ingredient or combination of ingredients.

For Control A, human dermal keratinocytes were left untreated. No irritation is expected, and therefore Control A provided a baseline. For Control B, IL-8 was induced in human dermal keratinocytes by applying a surfactant mixture that is a combination of sodium laureth sulfate and polyquaternium-10. For all other samples, the human dermal keratinocytes were co-treated with the surfactant mixture and a composition containing the ingredient of interest. Decreased IL-8 expression reflects the ingredient's anti-irritation activity. In other words, decreased IL-8 expression reflects reduced irritancy potential.

In order to carry out the test method, an assay kit was employed that was obtained from R&D Systems: Human CXCL8/IL-8 Duoset ELISA Kit.

The following steps were followed: 1. Coat EIA high binding 96-well plate overnight before use. 2. Prepare all reagents, standard dilutions, and samples. 3. Aspirate each well and wash the plate with 300 μL/well of wash buffer, repeating the process 3 times for a total of 4 washes. 4. Add 100 μL of Assay Diluent to each well. 5. Add 50 μL of Standard, control, or sample to each well. Cover with a plate sealer, and incubate at room temperature for 2 hours. 6. Aspirate each well and wash, repeating the process 3 times for a total of 4 washes. 7. Add 100 μL of Detection IL-8 Antibody to each well. Cover with a new plate sealer, and incubate at room temperature for 2 hour. 8. Aspirate and wash 4 times. 9. Add 100 μL of HRP conjugated Strepavidin solution in each well, incubating 20 minutes at room temperature. 10. Aspirate and wash 4 times. 11. Add 100 μL Substrate Solution to each well. Incubate at room temperature for 20 minutes, making sure to protect the wells from the light. 12. Add 50 μL of Stop Solution to each well. Using a colorimeter, absorbance of each well in the plates was measured at 450 nanometers (nm) within 30 minutes. Wavelength correction was set to 570 nm.

Aqueous solutions of acetyl hexapeptide-51 amide were prepared by dilution to achieve the concentrations shown in Table 1. Acetyl hexapeptide-51 amide was obtained from Lipotec under the tradename Juveleven.™ Juveleven.™ is a proprietary blend of butylene glycol, water, and acetyl hexapeptide-51 amide, containing approximately 0.05 wt. % acetyl hexapeptide-51 amide.

The concentration of acetyl hexapeptide-51 amide shown in the following table represents the concentration of the active ingredient. Thus, for example, in preparing Example 1A, 2 grams of Juveleven™ was mixed with 98 g deionized water to prepare a composition that was 2 wt % Juveleven™ and 10 parts per million by weight (ppm) acetyl hexapeptide-51 amide, based upon the total weight of the composition.

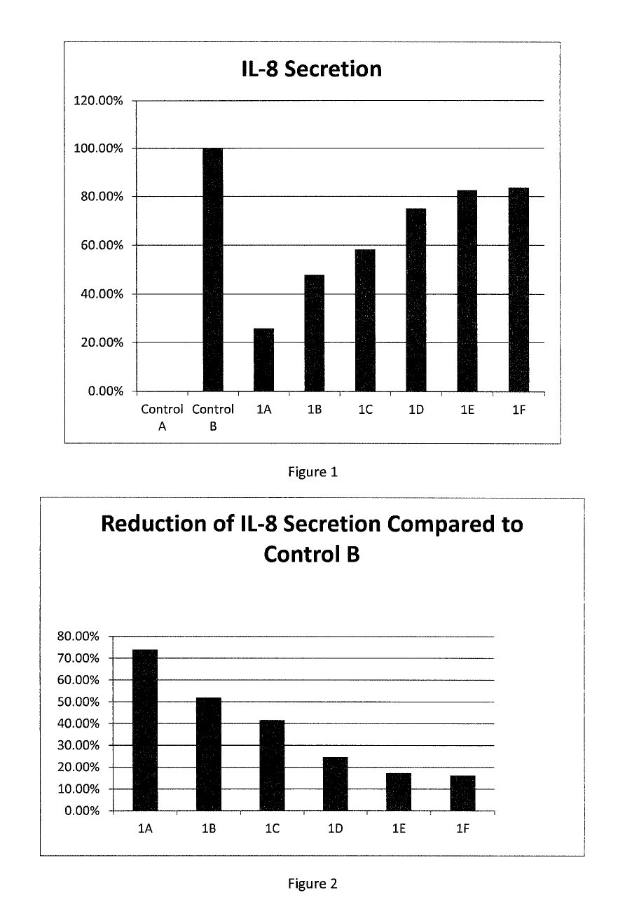

The samples were tested for IL-8 secretion as described in the test method above. That is, for Control A, human dermal keratinocytes were left untreated. For Control B, a surfactant mixture that was a combination of sodium laureth sulfate and polyquaternium-10 was employed. For all other samples, the human dermal keratinocytes were co-treated with the surfactant mixture and a composition containing varying amounts of acetyl hexapeptide-51 amide. Decreased 11-8 expression reflects the ingredient's anti-irritation activity. The results are summarized in Table 2 and shown graphically in FIG. 1. As can be seen in FIG. 1, acetyl hexapeptide-51 amide reduced IL-8 secretion, when compared to Control B.

The amount of the reduction in IL-8 secretion for the test samples (subtracting from 100% for Control B) is summarized in Table 2 and shown graphically in FIG. 2.

TABLE 1

| | Juveleven | Acetyl Hexapeptide- |
|---|---|---|
| Example | Wt. % | 51 amide ppm |
| 1A | 2 | 10 |
| 1B | 1 | 5 |
| 1C | 0.5 | 2.5 |
| 1D | 0.2 | 1 |
| 1E | 0.1 | 0.5 |
| 1F | 0.05 | 0.25 |

TABLE 2

| | | Reduction of | |
|---|---|---|---|
| | | IL-8 Secretion | |
| | | Compared to | Standard |
| Example | IL-8 secreted | Control B | Deviation |
| Control A | 0.00% | — | 2.10% |
| Control B | 100% | — | 13.0% |
| 1A | 25.90% | 74.10% | 4.70% |
| 1B | 47.90% | 52.10% | 8.70% |
| 1C | 58.40% | 41.60% | 9.90% |
| 1D | 75.20% | 24.80% | 18.40% |
| 1E | 82.60% | 17.40% | 20.00% |
| 1F | 83.7% | 16.30% | 23.80% |

Various modifications and alterations that do not depart from the scope and spirit of this invention will become apparent to those skilled in the art. This invention is not to be duly limited to the illustrative embodiments set forth herein.

What is claimed is:

1. A composition comprising at least one amide-functionalized oligopeptide.

2. The composition of claim 1, wherein the amide-functionalized oligopeptide includes 12 amino acid moieties or less.

3. The composition of claim 1, wherein the amide-functionalized oligopeptide includes 10 amino acid moieties or less.

4. The composition of claim 1, wherein the amide-functionalized oligopeptide includes 8 amino acid moieties or less.

5. The composition of claim 1, wherein the amide-functionalized oligopeptide includes at least 6 amino acid moieties.

6. The composition of claim 1, wherein the amide-functionalized oligopeptide is an amide-functionalized acetyl hexapeptide.

7. The composition of claim 1, wherein the functionalized acetyl hexapeptide is selected from the group consisting of acetyl hexapeptide-1, acetyl hexapeptide-7, acetyl hexapeptide-8, acetyl hexapeptide-19, acetyl hexapeptide-20, acetyl hexapeptide-22, acetyl hexapeptide-24, acetyl hexapeptide-30, acetyl hexapeptide-31, acetyl hexapeptide-37, acetyl hexapeptide-38, acetyl hexapeptide-39, and acetyl hexapeptide-46, acetyl hexapeptide-49, and acetyl hexapeptide-51.

8. The composition of claim 1, wherein the amide-functionalized acetyl hexapeptide is acetyl hexapeptide-51 amide.

9. The composition of claim 1, wherein the composition further comprises a cosmetically or pharmaceutically acceptable carrier.

10. The composition of claim 1, wherein the composition produces less skin irritation, when topically applied to mammalian skin, compared to the same composition but not including the amide-functionalized oligopeptide.

11. The composition of claim 9, wherein the carrier comprises:
at least one vegetal wax,
at least one olive derivative or extract, and
optionally, at least one emulsifier.

12. The composition of claim 11, wherein the vegetal wax is a blend of cetyl palmitate, sorbitan palmitate, and sorbitan olivate.

13. The composition of claim 11, wherein the olive derivative or extract is a blend of ceteraryl olivate and sorbitan olivate.

14. The composition of claim 11, wherein the emulsifier is glyceryl stearate.

15. The composition of claim 1, wherein the amount of amide-functionalized oligopeptide is from about 0.06 to about 100 ppm, based upon the total weight of the composition.

16. (canceled)

17. A method for reducing the irritancy potential of a skin cleanser or skin sanitizer composition, the method comprising the steps of:
combining a skin cleansing or skin sanitizer composition with one or more amide-functionalized oligopeptides, wherein the amide-functionalized oligopeptide reduces the irritancy potential of the composition.

18-20. (canceled)

21. A method for the cleansing and/or sanitizing skin, the method comprising the steps of:
combining a skin cleansing or skin sanitizing composition with one or more amide-functionalized oligopeptides, wherein the amide-functionalized oligopeptide reduces the irritancy potential of the composition; and
contacting the skin with the reduced-irritancy composition in a manner sufficient to cleanse and/or sanitize the skin.

22-33. (canceled)
